(19) United States
(12) Patent Application Publication
(10) Pub. No.: US 2021/0363543 A1
(43) Pub. Date: Nov. 25, 2021

(54) Self Replicating RNA System

(71) Applicant: NantBio, Inc., Culver City, CA (US)

(72) Inventors: Hermes J. Garban, Culver City, CA (US); Kayvan Niazi, Culver City, CA (US)

(21) Appl. No.: 17/281,555

(22) PCT Filed: Oct. 29, 2019
(86) PCT No.: PCT/US2019/058591

§ 371 Date: Mar. 30, 2021

Related U.S. Application Data
(60) Provisional application No. 62/752,824, filed on Oct. 30, 2018.

(51) Int. Cl. C12N 15/86; C07K 14/07; A61K 35/17; C07K 14/47; C07K 16/28

(52) U.S. Cl. CPC C12N 15/86; C07K 14/07; A61K 35/17; C07K 14/47; C12N 2770/36143; C07K 2317/622; C12N 2510/00; C07K 2317/51; C07K 2317/515; C07K 16/28

(57) ABSTRACT

Compositions, methods and uses of self-replicating RNA molecules that include a recombinant nucleic acid encoding a protein of interest such as a chimeric antigenic receptor or an antibody are presented. The self-replicating RNA molecule are introduced into an immune competent cell such that the chimeric antigenic receptor or the antibody are efficiently expressed in the immune competent cell while increasing the stability of the recombinant nucleic acid in the cell and reducing the potential integration of the recombinant nucleic acid into the genome of the immune competent cell.

This application claims priority to our copending US provisional patent application with the Ser. No. 62/752,824, which was filed Oct. 30, 2018, and which is incorporated by reference herein.

### FIELD OF THE INVENTION

The field of the invention is compositions and methods for immunotherapy, especially as it relates to use of genetically modified immune cells that contain a self-replicating RNA that is engineered to express a heterologous protein.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

RNA-based vectors have been used as a safe and efficient way to deliver genetic information into cells for heterologous expression of genetic materials, especially in cell-based therapeutic applications. However, RNA-based vectors are prone to degradation and the expression of the payloads in such vectors is often highly dependent on efficient delivery methods. One way to overcome these problems is to increase stability by incorporating non-natural or modified nucleotides or to increase the level of expression of the encoded proteins. For example, some researchers incorporated pseudouridine into a RNA-based vector that increases stability and translational efficiency from the RNA-based vector. (Kariko et al., Mol Ther. 2008 November; 16(11): 1833-1840). In another example, incorporation of a longer poly(A) tail, unmasked poly(A) tail with a free 3′ end or 2 sequential β-globin 3 untranslated regions cloned head to tail between the coding region enhanced stability and translational efficiency. However, such RNA-based vectors are often difficult to prepare.

More recently, self-replicating RNA has emerged as a new tool for stable and effective introduction of heterologous genes to be expressed in a cell. The self-replicating RNA is a single-stranded RNA that has both positive and negative polarities and encodes its own RNA replicating enzymes, which induces rapid and high quantity cytoplasmic RNA replication. Using such self-replicating RNA, RNA-based self-replicating vectors that encode various antigens as heterologous gene products by replacing structural genes have been generated to so express heterologous gene products without producing viral particles that can be toxic to the infected cells. For example, US Pat. Pub. No. 2014/0271829 to Lilja discloses expression of herpes virus proteins in the self-replicating RNA vector in the dendritic cells as to induce immune response against the herpes virus proteins.

Even though some uses of self-replicating RNA-based vector as a vaccine by expressing antigenic molecules in the infected cells are known, use of self-replicating RNA-based vector to genetically modify immune competent cells to more actively induce immune response against specific types of cells or specific types of antigen remained unexplored. Thus, there remains a need for improved compositions, methods for and uses of self-replicating RNA-based vectors in modifying immune competent cells other than antigen presenting cells.

### SUMMARY OF THE INVENTION

The inventive subject matter is directed to various compositions of, methods for, and use of a self-replicating RNA molecule in immune competent cells, and especially NK cells, in which the RNA encodes a chimeric antigen receptor or an antibody to express such molecules.

Thus, one aspect of the subject matter includes a self-replicating recombinant expression vector that is derived from a self-replicating RNA molecule. The self-replicating recombinant expression vector includes a recombinant nucleic acid encoding a chimeric antigen receptor. The chimeric antigen receptor has 1) an extracellular single-chain variant fragment that specifically binds a tumor neoepitope, tumor associated antigen, or self-lipid, 2) an intracellular activation domain, and 3) a transmembrane linker coupling the extracellular single-chain variant fragment to the intracellular activation domain. Preferably, the chimeric antigen receptor has a V_{L} domain and a V_{H} domain that are configured to bind to a tumor neoepitope, a tumor associated antigen, or a self-lipid.

In another aspect of the inventive subject matter, the inventors contemplate a genetically modified immune competent cell having a self-replicating RNA molecule. The self-replicating RNA molecule includes a recombinant nucleic acid encoding a chimeric antigen receptor. The chimeric antigen receptor has 1) an extracellular single-chain variant fragment that specifically binds a tumor neoepitope, tumor associated antigen, or self-lipid, 2) an intracellular activation domain, and 3) a transmembrane linker coupling the extracellular single-chain variant fragment to the intracellular activation domain. Preferably, the chimeric antigen receptor has a V_{L} domain and a V_{H} domain that are configured to bind to a tumor neoepitope, a tumor associated antigen, or a self-lipid. In some embodiments, the immune competent cell is one of a T cell, a NK cell, a NKT cell, or a genetically engineered NK cells (e.g., aNK, haNK, taNK cells, etc.).

Still another aspect of inventive subject matter is directed towards a self-replicating recombinant expression vector that includes a recombinant nucleic acid encoding an antibody. The recombinant nucleic acid includes a first nucleic acid segment encoding a heavy chain and a second nucleic acid segment encoding a light chain. Preferably, the first and second sets of nucleic acid segments are downstream of two distinct subgenomic promoters. The antibody, when expressed, is configured to bind a tumor-associated antigen, a cell surface molecule expressed on a tumor cell, a cytokine or a chemokine, or to a ligand of a T cell receptor, NK cell receptor, or a NKT cell receptor.

Still another aspect of inventive subject matter is directed towards a genetically modified immune competent cell that has a self-replicating RNA molecule including a recombinant nucleic acid encoding an antibody. The recombinant nucleic acid includes a first nucleic acid segment encoding a heavy chain and a second nucleic acid segment encoding a light chain. Preferably, the first and second sets of nucleic acid segments are downstream of two distinct subgenomic promoters. The antibody, when expressed, is configured to bind a tumor-associated antigen, a cell surface molecule expressed on a tumor cell, a cytokine or a chemokine, or to a ligand of a T cell receptor, NK cell receptor, or a NKT cell receptor. Preferably, the immune competent cell expression such antibody can be one of B cell, T cell, a NK cell, a NKT cell, or a genetically engineered NK cells (e.g., aNK, haNK, taNK cells, etc.).

In still another aspect of the inventive subject matter, the inventors contemplate a method of increasing effectiveness of immune therapy in a person having a tumor. In this method, a genetically modified immune competent cell that includes self-replicating RNA molecule is provided. The self-replicating RNA molecule includes a recombinant nucleic acid encoding a chimeric antigen receptor. The chimeric antigen receptor has 1) an extracellular single-chain variant fragment that specifically binds a tumor neoepitope, tumor associated antigen, or self-lipid, 2) an intracellular activation domain, and 3) a transmembrane linker coupling the extracellular single-chain variant fragment to the intracellular activation domain. Preferably, the chimeric antigen receptor has a V_{L} domain and a V_{H} domain that are configured to bind to a tumor neoepitope, a tumor associated antigen, or a self-lipid. The genetically modified immune competent cell can then be administered to the patient in a dose and a schedule effective to treat the tumor.

In still another aspect of the inventive subject matter, the inventors contemplate a method of increasing production of an antibody of interest in a patient. In this method, a genetically modified immune competent cell comprising a self-replicating RNA molecule is provided. The immune competent cell has a self-replicating RNA molecule, which includes a recombinant nucleic acid encoding an antibody. The recombinant nucleic acid includes a first nucleic acid segment encoding a heavy chain and a second nucleic acid segment encoding a light chain. Preferably, the first and second sets of nucleic acid segments are downstream of two distinct subgenomic promoters. The antibody, when expressed, is configured to bind a tumor-associated antigen, a cell surface molecule expressed on a tumor cell, a cytokine or a chemokine, or to a ligand of a T cell receptor, NK cell receptor, or a NKT cell receptor. Preferably, the immune competent cell expression such antibody can be one of B cell, T cell, a NK cell, a NKT cell, or a genetically engineered NK cells (e.g., aNK, haNK, taNK cells, etc.). The genetically modified immune competent cell can then be administered to the patient to produce specific antibodies in the patient's body.

Still another aspect of the inventive subject matter includes use of the genetically modified immune competent cell or self-replicating recombinant expression vector that includes a recombinant nucleic acid encoding a chimeric antigen receptor for increasing effectiveness of immune therapy in a person having a tumor. Further, another aspect of the inventive subject matter includes use of the genetically modified immune competent cell or self-replicating recombinant expression vector that includes a recombinant nucleic acid encoding a chimeric antigen receptor for increasing immune response in patient having a disease.

In still another aspect of the inventive subject matter, the inventors also contemplate use of the genetically modified immune competent cell or self-replicating recombinant expression vector that includes a recombinant nucleic acid encoding an antibody for increasing production of the antibody of interest in a patient.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic illustration of an alpha virus genome structure.

FIG. 2 is a schematic illustration of polyprotein processing and RNA synthesis of an alpha virus.

FIG. 3 is a schematic illustration of a recombinant Venezuelan Equine Encephalitis Virus (VEEV) based self-replicating RNA vector for heterologous protein expression according to the inventive subject matter.

### DETAILED DESCRIPTION

The inventors discovered that expression of functional, antigen-targeting heterologous recombinant molecules in immune competent cells can be achieved using a self-replicating RNA vector system to so create modified immune competent cells, and especially modified NK cells. Viewed from a different perspective, the inventors discovered that various recombinant RNA expression vectors, and genetically modified immune competent cells including such various recombinant RNA expression vectors, can be generated such that functional, antigen-targeting recombinant molecules can be effectively and at least transiently produced when the genetically modified immune competent cells are introduced into a subject. Such modified cells can advantageously be used in vivo to produce a desired, typically target-specific cytotoxic effect.

In this context it should be particularly noted that the recombinantly expressed molecules are not an immunogen nor are recombinant cells expressing the recombinant molecule used as a vaccine. In contrast, the recombinantly expressed molecules are used to guide immune competent cells to specific and desired antigens where the immune competent cells then exert their target-specific cytotoxic effects. Moreover, the inventors have also shown that an RNA-based expression approach in immune competent cells can be taken that (1) utilizes a single RNA species with self-replicative capacity for a limited number of cell divisions, thereby reducing the number of transfections, (2) encodes a polycistronic open reading frame (ORFs), (3) expresses robustly all encoded heterologous genes over multiple rounds of cell divisions, and (4) can be selectively retained or degraded in a controlled manner.

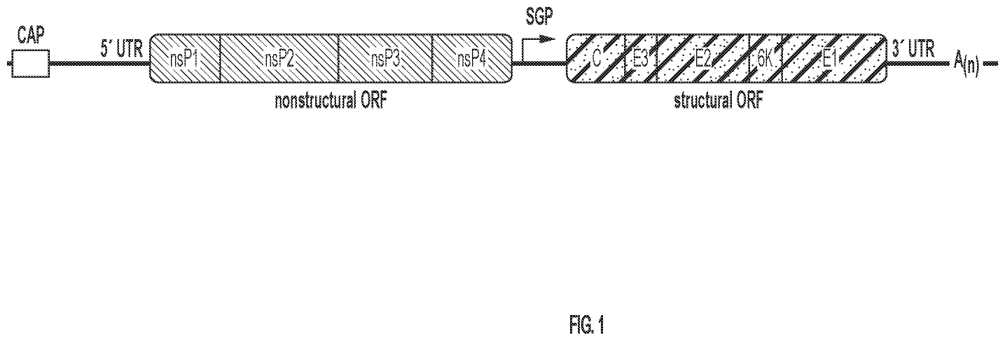

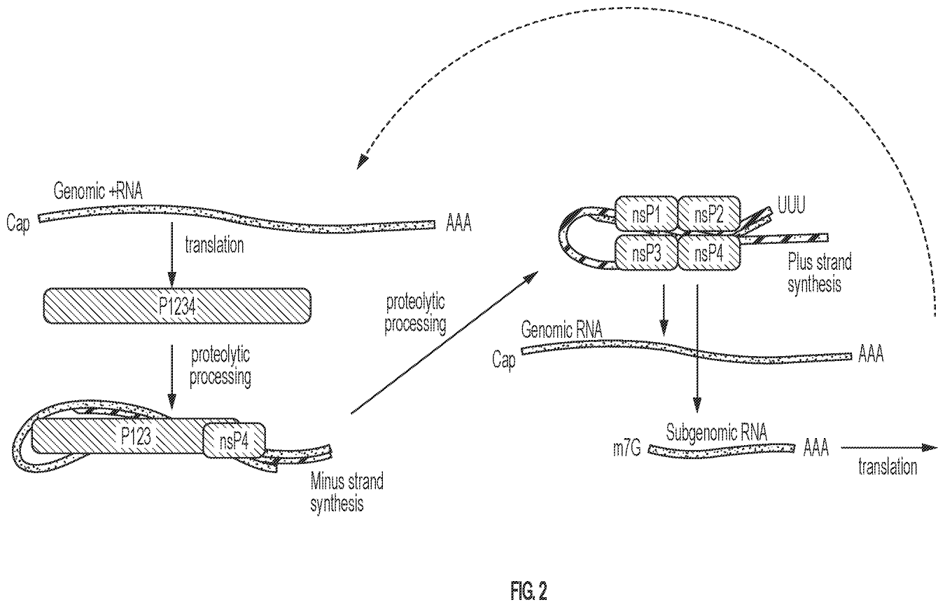

As can be seen from FIG. 1, a typical self-replicating RNA genome of an Alphavirus is depicted where the positive-sense RNA genome is about 11.5 kilobases in length and contains two open reading frames (ORF). The first ORF encodes the non-structural proteins (nsPs) 1-4, while the second ORF encodes structural proteins (C, capsid; E1/2/3, envelope glycoproteins and 6 K, a 6 kDa protein). UTR denotes the untranslated region, SGP denotes the subgenomic promoter, and A(n) denotes the polyA tail. FIG. 2 schematically depicts the polyprotein processing and RNA synthesis of the Alphavirus, which is typical for self-replicating RNA molecules. Here, the viral plus-strand RNA is released and non-structural proteins (nsPs) are translated as a polyprotein. After cleavage of nsP4, the replication complex synthesizes the minus strand from the genomic RNA. Further cleavage of all nsPs to individual proteins switches synthesis to genomic and subgenomic positive-strand RNA. The structural proteins are then translated from the subgenomic RNA, leading to the expression of the cargo protein(s) and the genomic +RNA strand will enter again into a new cycle of RNA synthesis.

As is shown in more detail below, and to express heterologous genes (e.g., Chimeric Antigen Receptors (CARs)), the inventors modified a non-infectious (non-packaging), self-replicating Venezuela equine encephalitis (VEE) virus RNA replicon system. This VEE-based replicon is a positive (+), single stranded RNA that resembles a cellular mRNA that contains a 5′ cap and poly(A) tail and that does not use a DNA intermediate. Consequently, there is no potential for genomic integration. Such self-replicative RNA-based expression system is preferably used for heterologous gene expression of various designed CARs, antibodies and antigen-binding fragments thereof (or any other protein or antigen) to transfect primary lymphocytes (e.g., B-cells, T-cells, myelo-derived cells, monocytes, Natural Killer cells, etc.) or NK-derived cell lines (i.e., aNKs, haNKs, etc). In addition, contemplated self-replicating systems could be used in methods aiming to produce naturally evolving variants of the newly generated subgenomic RNA segments, resulting in modified functional properties of the expressed products (e.g., binding affinity, etc). This property could be used in methods related to the improvement of binding affinity of antibodies (Abs) (e.g., Ab affinity maturation).

In one exemplary and especially preferred aspect of the inventive subject matter, the inventors contemplate a self-replicating recombinant expression vector having a recombinant nucleic acid encoding a chimeric protein, preferably a chimeric antigen receptor (CAR). Most typically, the self-replicating recombinant expression vector is an RNA molecule that is obtained or derived from an alphavirus (e.g., Semliki Forest virus, Sindbis virus, Venezuelan equine encephalitis virus, etc.). While it is less typical, it is also contemplated that the self-replicating recombinant expression vector is an RNA molecule that is derived from Flaviviruses (e.g., Kunjin virus, West Nile virus, yellow fever virus, dengue virus, tick-borne encephalitis virus, etc.), Measles Viruses, or Rhabdoviruses (e.g., Rabies virus, Vesicular stomatitis virus, etc.).

Where the self-replicating recombinant expression vector is alphavirus-based, it is contemplated that the self-replicating recombinant expression vector is generally a linear RNA molecule (a plus-strand RNA) that includes at least two open reading frames. The first open reading frame includes genes encoding nonstructural proteins (nsP1, nsP2, nsP3, nsP4), and the second open reading frame includes a gene of interest, which expression is driven by a subgenomic promoter located upstream of the second open reading frame. Preferably, the subgenomic promoter is an endogenous subgenomic promoter of the alphavirus. However, it is also contemplated that the subgenomic promoter is a heterologous promoter derived from other types of virus (e.g., cytomegalovirus, etc.) or other mammalian expression system. It is contemplated that translation of the nonstructural proteins in the first open reading frame generates an early replication complex formed by the polyprotein P123 and nsP4, which synthesizes a minus-strand RNA from a plus-strand RNA as a template. The late replication complex formed with fully processed nsP1-nsP4 generates genomic and subgenomic plus strands RNA from the minus-strand RNA as a template. Translation of such generated subgenomic plus strands RNA generates a heterologous peptide of the gene of interest. Thus, while the recombinant expression vector self-replicates by its own RNA synthase translated from the first open reading frame, the recombinant expression vector can continuously produce the heterologous peptides in the transfected cell.

The inventors further contemplate that, in some embodiments, the second open reading frame may include an enhancing element in the upstream (toward 5′-end) of the gene of interest. In some embodiments, the enhancing element includes a portion of genetic element of the first open reading frame that can increase the translation efficiency of the gene of interest. For example, where the self-replicating recombinant expression vector is alphavirus-based, the portion of genetic element includes a 51-nucleotide conserved sequence in nsP1-encoding region (nsP1 fragment). Thus, the portion of genetic element may include at least 10 nucleotides, 30 nucleotides, 50 nucleotides, or 100 nucleotides upstream (toward 5′-end) or downstream (toward 3′3nd) or both from the 51-nucleotide conserved sequence.

Additionally, the enhancing element also include a spacer nucleotide coupling the nsP1 fragment to the gene of interest such that the translated peptide from the gene of interest can be present without additional amino terminal peptides attached. In such embodiment, the nsP1, the spacer nucleotide, and the gene of interest are located preferably in the same reading frame. While any suitable nucleotide sequences in any suitable length are contemplated, exemplary nucleotide sequences includes nucleic acid sequences encoding ubiquitin, 2A protease of foot-and-mouth-disease virus (FMDV 2A), or VEEV capsid protein. In case where the VEEV capsid protein is used as a spacer nucleotide, it is preferred that the nucleotide encoding such VEEV capsid protein lacks nuclear localization signal (which inhibits transcription in the host cell) such that the toxicity of the VEEV capsid protein to the host cell is reduced.

In some embodiments, the second open reading frame can be polycistronic such that it includes two or more subgenomic promoters. In such system, two or more genes of interest (related or not) can be expressed concurrently upon translation of the second open reading frame. While the length (or sizes) of the subgenomic promoter and the genes of interest downstream of each subgenomic promoter may vary depending on the type of genes, it is preferred that the total length of the second open reading frame is no more than 15 kb, preferably no more than 10 kb, more preferably no more than 5 kb. Optionally, one or more subgenomic promoters can be coupled with an enhancing element described above such that expression of each gene of interest under such subgenomic promoter can be individually and/or independently enhanced.

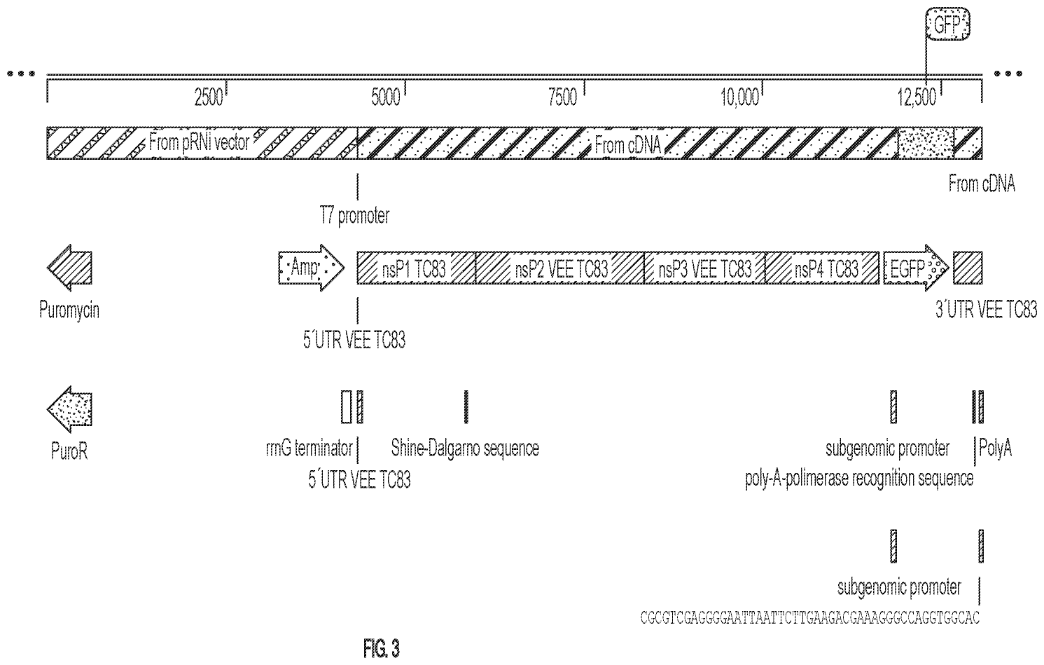

One exemplary recombinant VEEV-based self-replicating RNA vector for protein expression is schematically depicted in FIG. 3. Here, a DNA vector comprises a plasmid portion (here: pRNi vector) and a cDNA portion that corresponds to the recombinant VEEV RNA genome. As can be seen, the VEEV RNA genome portion is preceded by the T7 promotor to generate the corresponding +strand RNA for transfection. The VEEV RNA genome comprises a first ORF for the four non-structural proteins nsP1-T83, nsP2-VEE T83, nsP3-VEE T83, and nsP4-T83, and a second ORF that includes a recombinant protein (here: eGFP) that is under the control of a subgenomic promotor of the VEEV. Downstream of the second ORF is a 3′-UTR region of VEE TC83 and a polyA sequence.

### Chimeric Antigen Receptor (CAR) as a Gene of Interest

While any suitable gene of interest can be placed in the second open reading frame, the inventors contemplate that the gene of interest may include a nucleic acid sequence encoding a chimeric antigen receptor (CAR). CAR generally includes an extracellular single-chain variant fragment, an intracellular activation domain, and a transmembrane linker coupling the extracellular single-chain variant fragment to the intracellular activation domain. Preferably, the recombinant protein is generated from a single chimeric polypeptide translated from a single recombinant nucleic acid. However, it is also contemplated that that the recombinant protein comprises at least two domains that are separately translated from two distinct recombinant nucleic acid such that at least a portion of the recombinant protein can be reversibly coupled with the rest of the recombination protein via a protein-protein interaction motif.

Thus, in a preferred embodiment, in which the recombinant protein is encoded by a single recombinant nucleic acid, the recombinant nucleic acid includes at least three nucleic acid segments: a first nucleic acid segment (a sequence element) encoding an extracellular single-chain variant fragment that specifically binds to a target molecule; a second nucleic acid segment encoding an intracellular activation domain; and a third nucleic acid segment encoding a linker between the extracellular single-chain variant fragment and the intracellular activation domain.

In this embodiment, the first nucleic acid segment encoding an extracellular single-chain variant fragment includes a nucleic acid sequence encoding a heavy (V_{H}) and light chain (V_{L}) of an immunoglobulin. In a preferred embodiment, the nucleic acid sequence encoding variable regions of the heavy chain (V_{H}) and the nucleic acid sequence encoding variable regions of the light chain (V_{L}) are separated by a linker sequence encoding a short spacer peptide fragment (e.g., at least 10 amino acid, at least 20 amino acid, at least 30 amino acid, etc.). Most typically, the extracellular single-chain variant fragment encoded by the first nucleic acid segment includes one or more nucleic acid sequences that determine the binding affinity and/or specificity to the tumor neoepitope or a tumor associated antigen. Thus, the nucleic acid sequence of V_{H} and V_{L} can vary depending on sequence of the target molecule the recombinant protein may target to.

It is contemplated that the target molecule can be any peptide or lipid that is expressed by a cell or an organism that the immune response by the immune competent cell expressing the CAR is desired. Thus, the target molecule may include, but not limited to, a tumor associated antigen, a patient-, tumor-specific neoepitope, or self-lipid presented on the tumor cell surface, a viral peptide, a peptide derived from any other pathogens (e.g., yeast, bacteria, etc.).

Any suitable methods to identify the nucleic acid sequence of V_{H} and V_{L} specific to the target molecules are contemplated. For example, a nucleic acid sequence of V_{H} and V_{L} can be identified from a monoclonal antibody sequence database with known specificity and binding affinity to the target molecule. Alternatively, the nucleic acid sequence of V_{H} and V_{L} can be identified via an in silico analysis of candidate sequences (e.g., via IgBLAST sequence analysis tool, etc.). In some embodiments, the nucleic acid sequence of V_{H} and V_{L} can be identified via a mass screening of peptides having various affinities to the target molecule via any suitable in vitro assays (e.g., flow cytometry, SPR assay, a kinetic exclusion assay, etc.). While it may vary depending on the characteristics of the target molecule, it is preferred that the optimal nucleic acid sequence of V_{H} and V_{L} encodes an extracellular single-chain variant fragment having an affinity to the tumor epitope at least with a KD of at least equal or less than 10⁻⁶M, preferably at least equal or less than 10⁻⁷M, more preferably at least equal or less than 10⁻⁸M. Alternatively, synthetic binders to the tumor epitope may also be obtained by phage panning or RNA display.

While it is preferred that that the first nucleic acid segment includes nucleic acid sequences encoding one of each heavy (V_{H}) and light chains (V_{L}), it is also contemplated that in some embodiments, the first nucleic acid segment includes nucleic acid sequence encoding a plurality of heavy (V_{H}) and light chains (V_{L}) (e.g., two heavy (V_{H}) and light chains (V_{L}) for generating a divalent (or even a multivalent) single-chain variable fragments (e.g., tandem single-chain variable fragments). In this embodiment, the sequence encoding one of each heavy (V_{H}) and light chains (V_{L}) can be linearly duplicated (e.g., V_{H}-linker 1-V_{L}-linker 2-V_{H}-linker 3-V_{L}). It is contemplated that the length of the linkers 1, 2, 3 can be substantially similar or same. However, it is also contemplated that the length of linker 2 is substantially different (e.g., longer or shorter) than the length of linker 1 and/or linker 3.

The recombinant nucleic acid also includes a second nucleic acid segment (a sequence element) encoding an intracellular activation domain of the recombinant protein. Most typically, the intracellular activation domain includes one or more ITAM activation motifs (immunoreceptor tyrosine-based activation motif, YxxL/I-X6-8-YXXL/I), which triggers signaling cascades in the cells expressing these motifs. Any suitable nucleic acid sequences including one or more ITAM activation motifs are contemplated. For example, the sequence of the activation domain can be derived from a NK receptor including one or more ITAM activation motif (e.g., intracellular tail domain of killer activation receptors (KARs), NKp30, NKp44, and NKp46, etc.). In another example, the sequence of the activation domain can be derived from a tail portion of a NKT T-cell antigen receptor (e.g., CD3ζ, CD28, etc.). In some embodiments, the nucleic acid sequence of the intracellular activation domain can be modified to add/remove one or more ITAM activation motif to modulate the cytotoxicity of the cells expressing the recombinant protein.

The first and second nucleic acid segments are typically connected via a third nucleic acid segment encoding a linker portion of the recombinant protein. Preferably, the linker portion of the recombinant protein includes at least one transmembrane domain. Additionally, the inventors contemplate that the linker portion of the recombinant protein further includes a short peptide fragment (e.g., spacer with a size of between 1-5 amino acids, or between 3-10 amino acids, or between 8-20 amino acids, or between 10-22 amino acids) between the transmembrane domain and the extracellular single-chain variant fragment, and/or another short peptide fragment between the transmembrane domain and the intracellular activation domain. In some embodiments, the nucleic acid sequence of transmembrane domain and/or one or two short peptide fragment(s) can be derived from the same or different molecule from which the sequence of intracellular activation domain is obtained.

In other contemplated embodiments, the recombinant nucleic acid includes a nucleic acid segment encoding a signaling peptide that directs the recombinant protein to the cell surface. Any suitable and/or known signaling peptides are contemplated (e.g., leucine rich motif, etc.). Preferably, the nucleic acid segment encoding an extracellular single-chain variant fragment is located in the upstream of the first nucleic acid segment encoding an extracellular single-chain variant fragment such that the signal sequence can be located in N-terminus of the recombinant protein. However, it is also contemplated that the signaling peptide can be located in the C-terminus of the recombinant protein, or in the middle of the recombinant protein.

### Antibody as a Gene of Interest

The inventors also contemplate that the gene of interest may include a nucleic acid sequence encoding an antibody binding to a target protein. Where the antibody is an immunoglobulin, it is contemplated that the immunoglobulin can include any type (e.g., IgG, IgE, IgM, IgD, IgA and IgY) and any class (e.g., IgG₁, IgG₂, IgG₃, IgG₄, IgA₁ and IgA₂) of heavy chain or constant domain to constitute different types of immunoglobulin. In addition, the “antibody” can include, but not limited to a human antibody, a humanized antibody, a chimeric antibody, a monoclonal antibody, a polyclonal antibody. Thus, while any types of antibodies are contemplated, it is generally preferred that the antibody expressed using the self-replicating RNA system is an IgG by its structural simplicity and smaller size than other types of antibodies (e.g., IgM, IgA, etc.). In such embodiment, the nucleic acid sequence includes a first nucleic acid segment encoding a heavy chain and a second nucleic acid segment encoding a light chain, which are placed separately at the downstream of two separate subgenomic promoters in the polycistronic second open reading frame.

The nucleic acid sequence encoding each segment may vary depending on the target molecule, modification of constant region, and types of antibody (membrane-bound or soluble). For example, nucleic acid sequence encoding variable regions of the heavy chain (V_{H}) and the nucleic acid sequence encoding variable regions of the light chain (V_{L}) may be selected based on the affinity and specificity to the target molecule. Again, any suitable methods to identify the nucleic acid sequence of V_{H} and V_{L} specific to the target molecules are contemplated. For example, a nucleic acid sequence of V_{H} and V_{L} can be identified from a monoclonal antibody sequence database with known specificity and binding affinity to the target molecule. Alternatively, the nucleic acid sequence of V_{H} and V_{L} can be identified via an in silico analysis of candidate sequences (e.g., via IgBLAST sequence analysis tool, etc.). In some embodiments, the nucleic acid sequence of V_{H} and V_{L} can be identified via a mass screening of peptides having various affinities to the target molecule via any suitable in vitro assays (e.g., flow cytometry, SPR assay, a kinetic exclusion assay, etc.). While it may vary depending on the characteristics of the target molecule, it is preferred that the optimal nucleic acid sequence of V_{H} and V_{L} encodes an extracellular single-chain variant fragment having an affinity to the tumor epitope at least with a K_{D} of at least equal or less than 10⁻⁶M, preferably at least equal or less than 10⁻⁷M, more preferably at least equal or less than 10⁻⁸M. Alternatively, synthetic binders to the tumor epitope may also be obtained by phage panning or RNA display.

It is preferred that the nucleic acid sequence encoding the constant region of the heavy chain (C_{H1}, C_{H2}, C_{H3}) and/or constant region of the light chain (CL) can be individual-specific (e.g., patient-specific). In such embodiment, it is preferred that the nucleic acid sequence encoding the constant region of the heavy chain can be at least 80%, at least 90%, or at least 95% homologous to one or more IgG identified or isolated from the individual such that the effector function of the antibody can be optimized to such individual. In some embodiments, the inventors contemplate that the effector function of the antibody can be augmented by introducing one or more amino acid mutations in one of the C_{H2} and C_{H3} region (Fc domain) that can increase antibody-mediated cellular cytotoxicity (ADCC) or antibody-dependent cell-mediated phagocytosis (ADCP). For example, the Fc domain of antibody may carry K326W/E333S mutations that can increase antibody-mediated cell lysis by several folds.

It is also contemplated that the antibody encoded by the self-replicating RNA system may be a membrane-bound form of an antibody (surface immunoglobulin), which triggers B cell activation upon binding to the antigen, or a soluble form of an antibody that can secreted from the cell. Thus, in some embodiments, the nucleic acid segment encoding the heavy chain of the antibody may further include a transmembrane domain at its C-terminus such that the assembled antibody with the heavy chain can be expressed on the cell membrane. In other embodiments, the nucleic acid sequence includes a first nucleic acid segment encoding a heavy chain without a transmembrane domain at its C-terminus, a second nucleic acid segment encoding a heavy chain with a transmembrane domain at its C-terminus, and a third nucleic acid segment encoding a light chain (and optionally a fourth nucleic acid segment encoding a light chain), which are placed separately at the downstream of three (or optionally four, where the fourth nucleic acid segment is present) separate subgenomic promoters in the polycistronic second open reading frame such that both soluble form and the membrane bound form of antibodies can be generated from the single self-replicating RNA system. Alternatively, it is also contemplated that two separate and distinct self-replicating recombinant expression vectors can be generated such that one self-replicating recombinant expression vector includes a first nucleic acid segment encoding a heavy chain with a transmembrane domain at its C-terminus and a second nucleic acid segment encoding a light chain, and another self-replicating recombinant expression vector includes a first nucleic acid segment encoding a heavy chain without a transmembrane domain at its C-terminus and a second nucleic acid segment encoding a light chain.

It is contemplated that the target molecule can be any peptide, lipid, a glycolipid, or a carbohydrate chain that is expressed by a cell or an organism and/or present in the specific disease conditions (e.g., tumor microenvironment, etc.) that inhibition of activity or interaction of such molecule is desired. Thus, the target molecule may include, but not limited to, a tumor associated antigen, a patient-, tumor-specific neoepitope, or self-lipid presented on the tumor cell surface, a viral peptide, a peptide derived from any other pathogens (e.g., yeast, bacteria, etc.), a cytokine or a chemokine (e.g., IL-2, IL-15, TGF-β, etc.), a ligand of a T cell receptor, NK cell receptor, or a NKT cell receptor, a receptor of Treg cells, or any other types of cell-membrane bound molecule or cell-secreted molecule.

Such generated self-replicating RNA-based vector can then be delivered to the any immune competent cells to so stably and effectively express the gene of interest in the immune competent cells. As used herein, the immune competent cell refers any cells in the immune system that can play a role in inducing an immune response to a foreign or self-antigen. Thus, the immune competent cell includes T cells, B cells, NK cells, NKT cells, dendritic cells, macrophage and any subtypes thereof. In some embodiments, the immune competent cell is an autologous immune competent cell that is obtained/isolated from an individual (e.g., patient) and optionally expanded ex vivo. Yet, it is also contemplated that the immune competent cell can be obtained from genetically engineered cell line, for example, NK92 cells and derivatives thereof (e.g., aNK cells, haNK cells, taNK cells, all commercially available from NantKwest, 9920 Jefferson Blvd. Culver City, Calif. 90232).

Any suitable delivery methods for introducing the self-replicating RNA-based vector to the immune competent cells are contemplated. In one embodiments, the self-replicating RNA-based vector can be formulated with a pharmaceutically acceptable carrier (e.g., as a sterile injectable composition (e.g., buffer, preferably with RNAase inhibitors)) such that the self-replicating RNA-based vector can be delivered in a naked form to the cell by contacting directly to the cell membrane. In some embodiments, the self-replicating RNA-based vector can be coupled to a carrier molecule. Exemplary carrier molecules includes protein A, protein G, protein Z, albumin, refolded albumin, a nanoparticle (e.g., quantum dots, gold nanoparticles, magnetic nanoparticles, nanotubes, polymeric nanoparticles, dendrimers, etc.), or a bead (e.g., polystyrene bead, latex bead, dynabead, etc.). Preferably, the nanoparticle and/or beads have a dimension below 1 μm, preferably below 100 nm. In other embodiments, the self-replicating RNA-based vector can be coupled with a microparticle (e.g., PLG RG503 (50:50 lactide/glycolide molar ratio), where the self-replicating RNA-based vector can be absorbed to the microparticle for further delivery to the cell. In still other embodiments, the self-replicating RNA-based vector can be encapsulated in a liposome (e.g., PEG-based liposome, etc.) to protect the self-replicating RNA-based vector from RNAase digestion and deliver the RNA-based vector by fusing the liposome to the target cell membrane.

In some embodiments, the inventors contemplate that the self-replicating RNA-based vector can be directly administered to the patient such that the self-replicating RNA-based vector can be introduced to the cells, preferably immune competent cells, in vivo. As used herein, the term “administering” refers to both direct and indirect administration of the compounds and compositions contemplated herein, where direct administration is typically performed by a health care professional (e.g., physician, nurse, etc.), while indirect administration typically includes a step of providing or making the compounds and compositions available to the health care professional for direct administration. In such embodiment, it is preferred that the self-replicating RNA-based vector is encapsulated in a liposome (e.g., PEG-based liposome, etc.) to protect the self-replicating RNA-based vector from RNAase digestion in vivo, and such liposome is further associated with a directing molecule that can target the liposome to specific types of immune competent cells. For example, where the self-replicating RNA-based vector is desired to be introduced to T cells, the liposome may be associated with a binding molecule (e.g., a scFv molecule) to CD3. For other example, where the self-replicating RNA-based vector is desired to be introduced to NK cells, the liposome may be associated with a binding molecule to CD16 or CD56. Such binding molecules may be associated the liposome via a PEG moiety, which is incorporated into the lipid layer of the liposome.

In other embodiments, the inventors contemplate that the self-replicating RNA-based vector can be contacted with the immune competent cells (homogeneous or heterogeneous, autologous or non-autologous, optionally expanded and/or activated ex vivo) ex vivo to generate genetically modified immune competent cells having the self-replicating RNA-based vector in the cytoplasm. For example, NK cells isolated from an individual can be expanded ex vivo by incubating the isolated NK cells with using various activating molecules added in the culture media including cytokines (e.g., IL-2, IL-15, etc.), monoclonal antibodies (e.g., murine monoclonal antibody against CD3 (OKT3™), etc.), or using cell-to-cell interaction with activating cells (e.g., K562 cells, a cell line derived from a patient with myeloid blast crisis of chronic myelogenous leukemia and bearing the BCR-ABL1 translocation, etc.). Such expanded NK cells can be further incubated with self-replicating RNA-based vector (preferably associated with a nanoparticle carrier) for at least 2 hours, 6 hours, 12 hours, or 24 hours in 37° C. or 39° C. Such generated genetically modified immune competent cells can be further formulated with a pharmaceutically acceptable carrier (e.g., a buffer for a sterile injectable composition) and administered to the individual. In some embodiments, the genetically modified immune competent cell formulation can be administered via systemic injection including subcutaneous, subdermal injection, or intravenous injection. In other embodiments, where the systemic injection may not be efficient (e.g., for brain tumors, etc.), it is contemplated that the ex vivo activated NK cells and/or NKT cell formulation is administered via intratumoral injection.

The inventors further contemplate that replication of the self-replicating RNA-based vector and/or expression of the gene of interest carried by the self-replicating RNA-based vector can be optionally regulated such that the expression of the gene of interest can be maintained or tapered off. While any suitable chemical or biological mechanisms to retain/remove the self-replicating RNA-based vector or to facilitate/inhibit translation of the gene of interest in the host cell are contemplated, one exemplary embodiment may include co-expressing B18R in the immune competent cells. The inventors contemplate that, without wishing to be bound to any specific theory, the immune competent cells expressing B18R, or exposed to B18R, which is a decoy receptor for Type I Interferons (e.g., IFNα, IFNβ, etc.), can maintain the expression of the gene of interest at least 50%, preferably at least 60%, more preferably at least 70% of the initial expression level of the gene of interest after delivering self-replicating RNA-based vector to the immune competent cells, possibly by maintaining at least 30%, preferably at least 50%, more preferably at least 70% of replicated RNA molecules generated from the original self-replicating RNA-based vector.

Consequently, it is contemplated that the immune competent cell including the self-replicating RNA-based vector may also include a nucleic acid sequence encoding B18R. In some embodiments, the immune competent cells are co-transfected or sequentially transfected with the self-replicating RNA-based vector and a nucleic acid encoding B18R (preferably mRNA encoding B18R). In other embodiments, the self-replicating RNA-based vector may include mRNA encoding B18R in the second open reading frame under another sub-genome promoter such that B18R can be expressed concurrently with other gene of interest upon introduction to the immune competent cell.

Alternatively, the self-replicating RNA-based vector and/or the genetically modified immune competent cells having the self-replicating RNA-based vector can be administered to the patient with B18R as an adjuvant. In such embodiment, B18R can be concurrently administered with the self-replicating RNA-based vector and/or the genetically modified immune competent cells, or at least 3 hours, at least 6 hours, at least 12 hours, at least 24 hours after administering the self-replicating RNA-based vector and/or the genetically modified immune competent cells. In some embodiments where the expression of the gene of interest is desired to persist more than 3 days or more than 7 days, it is preferred that B18R can be administered systemically or locally at least once a day, at least once in 3 days, at least once in 5 days, etc. In other embodiments, where the expression of the gene of interest is desired to be tapered off, it is contemplated that the B18R administration can be stopped or gradually decreased over time until the expression of the gene of interest is lower than 20%, preferably lower than 10%, more preferably lower than 5% of the initial expression level or peak expression level of the gene of interest after administration of the self-replicating RNA-based vector and/or the genetically modified immune competent cells.

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “bind” refers to, and can be interchangeably used with a term “recognize” and/or “detect”, an interaction between two molecules with a high affinity with a K_{D} of equal or less than 10⁻⁶M, or equal or less than 10⁻⁷M. As used herein, the term “provide” or “providing” refers to and includes any acts of manufacturing, generating, placing, enabling to use, or making ready to use.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

1. A genetically modified immune competent cell, comprising the expression vector of claim 9, wherein the expression vector comprises
a self-replicating RNA molecule that includes a recombinant nucleic acid; and
wherein the recombinant nucleic acid encodes a chimeric protein having 1) an extracellular single-chain variant fragment that specifically binds a tumor neoepitope, tumor associated antigen, or self-lipid, 2) an intracellular activation domain, and 3) a transmembrane linker coupling the extracellular single-chain variant fragment to the intracellular activation domain, and
wherein the recombinant nucleic acid further encodes B18R.

2-8. (canceled)

9. An expression vector for production of a self-replicating recombinant RNA, comprising:
a recombinant nucleic acid that encodes a chimeric protein having 1) an extracellular single-chain variant fragment that specifically binds a tumor neoepitope, tumor associated antigen, or self-lipid, 2) an intracellular activation domain, and 3) a transmembrane linker coupling the extracellular single-chain variant fragment to the intracellular activation domain;
a recombinant nucleic acid that encodes B18R; and
wherein the recombinant expression vector includes a portion of a self-replicating RNA molecule.

10. The expression vector of claim 9, wherein the self-replicating expression vector includes a portion of an alphavirus selected from a group consisting of Semliki Forest virus, Sindbis virus, Venezuelan equine encephalitis virus.

11. The expression vector of claim 9, wherein the recombinant nucleic acid comprises:
a first nucleic acid segment encoding an extracellular single-chain variant fragment that specifically binds the tumor neoepitope, the tumor associated antigen, or the self-lipid;
a second nucleic acid segment encoding the intracellular activation domain;
a third nucleic acid segment encoding the linker between the extracellular single-chain variant fragment and the intracellular activation domain; and
wherein the first, second, and third segments are arranged such that the extracellular single-chain variant fragment, the intracellular activation domain, and the linker form a single chimeric polypeptide.

12. The expression vector of claim 9, wherein the extracellular single-chain variant fragment comprises a V_{L} domain and a V_{H} domain of a monoclonal antibody against a tumor neoepitope, a tumor associated antigen, or a self-lipid.

13. The expression vector of claim 12, further comprising a spacer between the V_{L} domain and the V_{H} domain.

14. The expression vector of claim 9, wherein a number of replication cycles of the self-replicating RNA molecule is controllable by addition or withdrawal of a controlling molecule.

15. The expression vector of claim 14, wherein the controlling molecule is a human fibroblast protein.

16. A genetically modified immune competent cell comprising the expression vector of claim 24, wherein the expression vector comprises a self-replicating RNA molecule that includes a recombinant nucleic acid encoding an antibody, and a recombinant nucleic acid encoding B18R.

17-23. (canceled)

24. An expression vector for production of a self-replicating recombinant RNA, comprising:
a recombinant nucleic acid that encodes an antibody, wherein the recombinant nucleic acid comprises:
a first nucleic acid segment encoding a heavy chain;
a second nucleic acid segment encoding a light chain; and
wherein the first and second sets of nucleic acid segments are downstream of two distinct subgenomic promoters; and
a recombinant nucleic acid that encodes B18R.

25. The expression vector of claim 24, wherein the self-replicating RNA expression vector is derived from an alphavirus selected from a group consisting of Semliki Forest virus, Sindbis virus, Venezuelan equine encephalitis virus.

26. The expression vector of claim 24, wherein the antibody is an IgG.

27. The expression vector of claim 24, wherein the antibody is configured to bind to a tumor-associated antigen.

28. The expression vector of claim 24, wherein the antibody is configured to bind to a cell surface molecule expressed on a tumor cell.

29. The expression vector of claim 24, wherein the antibody is configured to bind to a cytokine or a chemokine.

30. The expression vector of claim 24, wherein the antibody is configured to bind to a ligand of a T cell receptor, NK cell receptor, or a NKT cell receptor.

31. A method of increasing effectiveness of immune therapy in a person having a tumor, comprising:
providing the genetically modified immune competent cell of claim 1, comprising:
a self-replicating RNA molecule that includes a recombinant nucleic acid;
wherein the recombinant nucleic acid encodes a chimeric protein having 1) an extracellular single-chain variant fragment that specifically binds a tumor neoepitope, tumor associated antigen, or self-lipid, 2) an intracellular activation domain, and 3) a transmembrane linker coupling the extracellular single-chain variant fragment to the intracellular activation domain,
wherein the recombinant nucleic acid further encodes B18R; and
administering the genetically modified immune competent cell to the patient in a dose and a schedule effective to treat the tumor.

32-42. (canceled)

43. A method of increasing production of an antibody of interest in a patient, comprising:
providing a genetically modified immune competent cell comprising a self-replicating RNA molecule that includes a recombinant nucleic acid encoding an antibody, and a recombinant nucleic acid encoding B18R; and
administering the genetically modified immune competent cell to the patient.

44. The method of claim 43, wherein the self-replicating RNA molecule is derived from an alphavirus selected from a group consisting of Semliki Forest virus, Sindbis virus, Venezuelan equine encephalitis virus.

45. The method of claim 43, wherein the antibody is an IgG.

46. The method of claim 43, wherein the genetically modified immune competent cell is a B cell, T cell, a NK cell, or a NKT cell.

47. The method of claim 43, wherein the recombinant nucleic acid comprises:
a first set of nucleic acid segments encoding a heavy chain;
a second set of nucleic acids segments encoding a light chain;
wherein the first and second sets of nucleic acid segments are downstream of two distinct subgenomic promoter.

48. The method of claim 43, wherein the antibody is configured to bind to a tumor-associated antigen.

49. The method of claim 43, wherein the antibody is configured to bind to cell surface molecule expressed on a tumor cell.

50. The method of claim 43, wherein the antibody is configured to bind to a cytokine or a chemokine.

51. The method of claim 43, wherein the antibody is configured to bind to a ligand of a T cell receptor, NK cell receptor, or a NKT cell receptor.

52. The method of claim 43, wherein the genetically modified immune competent cell is generated by introducing a self-replicating recombinant expression vector to an immune competent cell, the self-replicating recombinant expression vector, comprising:
a recombinant nucleic acid encodes an antibody, wherein the recombinant nucleic acid comprises:
a first set of nucleic acid segments encoding a heavy chain;
a second set of nucleic acids segments encoding a light chain;
wherein the first and second sets of nucleic acid segments are downstream of two distinct subgenomic promoter.

53. The method of claim 43, further comprising controlling the replication of the self-replicating RNA molecule by administering a controlling molecule.

54. The method of claim 53, wherein the controlling molecule is a human fibroblast protein.

55-57. (canceled)
